(19) United States
(12) Patent Application Publication
(10) Pub. No.: US 2005/0187244 A1
(43) Pub. Date: Aug. 25, 2005

(54) Montelukast sodium polymorphs

(75) Inventors: Valerie Niddam-Hildesheim, Ein Vered (IL); Judith Aronhime, Rehovot (IL); Greta Sterimbaum, Rishon-Lezion (IL)

Correspondence Address: KENYON & KENYON, ONE BROADWAY, NEW YORK, NY, 10004, US

(73) Assignee: Entire Interest.

(21) Appl. No.: 11/048,283

(22) Filed: Jan. 31, 2005

Related U.S. Application Data
(60) Provisional application No. 60/540,567, filed on Jan. 30, 2004.

(51) Int. Cl. A61K 31/47; C07D 215/16

(52) U.S. Cl. 514/311; 546/178

(57) ABSTRACT

The present invention provides high crystallinity montelukast sodium, crystalline forms of montelukast sodium, and processes of preparing the same.

This application claims the benefit of U.S. Provisional patent application Ser. No. 60/540,567 filed Jan. 30, 2004.

### FIELD OF THE INVENTION

The present invention relates to the solid state chemistry of montelukast sodium.

### BACKGROUND OF THE INVENTION

Montelukast is a selective, orally active leukotriene receptor antagonist that inhibits the cysteinyl leukotriene CysLT₁ receptor. Leukotrienes are associated with the inflammation and constriction of airway muscles and the accumulation of fluid in the lungs. Montelukast sodium is a useful therapeutic agent for treating respiratory diseases such as asthma and allergic rhinitis.

The chemical name for montelukast sodium is: [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid, monosodium salt. Montelukast sodium is a hygroscopic, optically active, white to off-white powder. Montelukast sodium is freely soluble in methanol, ethanol, and water and practically insoluble in acetonitrile.

Montelukast sodium salt is represented by the formula:

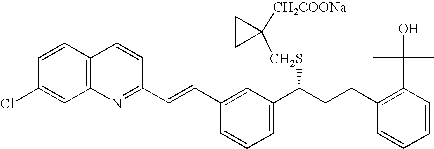

U.S. Pat. No. 5,565,473 discloses a synthetic process for montelukast sodium, wherein the compound is obtained as an oil that is then dissolved in water and freeze-dried.

The amorphous form of montelukast sodium is disclosed in U.S. Pat. No. 6,320,052 and WO 03/066598. The '052 patent discloses that the amorphous form is “not ideal for pharmaceutical formulation.” The '052 patent also discloses that the available processes for crystallizing montelukast sodium are “not particularly suitable for large-scale production” because of the “tedious chromatographic purification” technique required and because the “product yields are low.” The '052 patent discloses that in available processes, the free acids are converted directly to the corresponding sodium salt. The process of the '052 patent crystallizes montelukast sodium salt from a solution of montelukast in toluene and water and then acetonitrile (ACN) with seeding. Seeding is the use of a small amount of crystalline montelukast to induce crystallization in a larger sample. The crystalline form of the '052 patent has a low crystallinity index of less than about 30%.

The present invention relates to the solid state physical properties of montelukast sodium. These properties can be influenced by controlling the conditions under which montelukast sodium is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs, and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA), and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry, and infrared spectrometry.

Generally, the crystalline solid has improved chemical and physical stability over the amorphous form, and forms with low crystallinity. Since the crystallization process tends to remove impurities, the crystalline solid tends to have reduced levels of impurities over the amorphous form.

There is a need for high crystallinity montelukast sodium and additional forms of crystalline montelukast sodium. There is a need for improved methods for crystallizing montelukast sodium.

### SUMMARY OF THE INVENTION

In one embodiment, the present invention provides montelukast sodium having a crystalline index of at least about 40%, more preferably at least about 60%, more preferably at least about 70%, and most preferably at least about 80%. In a preferred embodiment, the crystalline montelukast sodium has a powder XRD pattern substantially free of peaks at 4.5 and 6.2±0.2 degrees two-theta. The present invention also provides solvates and hydrates of crystalline montelukast sodium.

In another embodiment, the present invention provides a process for preparing crystalline montelukast sodium comprising crystallizing the crystalline form from a solution of montelukast in a polar solvent and recovering the crystalline form. In a preferred embodiment, the polar solvent is aprotic. In another preferred embodiment, the polar solvent includes at least one of dimethyl carbonate, methyl isobutyl ketone, dichloromethane, dichloroethane, ethyl acetate, butyl acetate, isobutyl acetate, or water. In another embodiment, the process further comprises combining an anti-solvent with the solution. The anti-solvent is preferably a C₅ to C₁₂ hydrocarbon, most preferably heptane or hexane.

In another embodiment, the present invention provides crystalline forms of montelukast sodium named A1, B2, B1 C, D, and E. The present invention also provides processes of preparing these crystalline forms.

The present invention also provides pharmaceutical compositions comprising the crystalline montelukast sodium. The present invention also provides a method of treating asthma utilizing the same.

### BRIEF DESCRIPTION OF THE FIGURES

FIG. 1 depicts the calculation of crystalline index and consists of:

a) Baseline for calculation of the areas A_{C}+A_{A}, and A_{C} from the pattern in FIG. 3 of U.S. Pat. No. 6,320,052.
b) Example for calculation of crystalline index of high crystallinity montelukast of the present invention.

FIG. 2 depicts the X-ray powder diffraction pattern for montelukast sodium Form A1.

FIG. 3 depicts the X-ray powder diffraction pattern for montelukast sodium Form B2.

FIG. 4 depicts the X-ray powder diffraction pattern for montelukast sodium Form B1.

FIG. 5 depicts the X-ray powder diffraction pattern for montelukast sodium Form C.

FIG. 6 depicts the X-ray powder diffraction pattern for montelukast sodium Form D.

FIG. 7 depicts the X-ray powder diffraction pattern for montelukast sodium Form E.

### DETAILED DESCRIPTION OF THE INVENTION

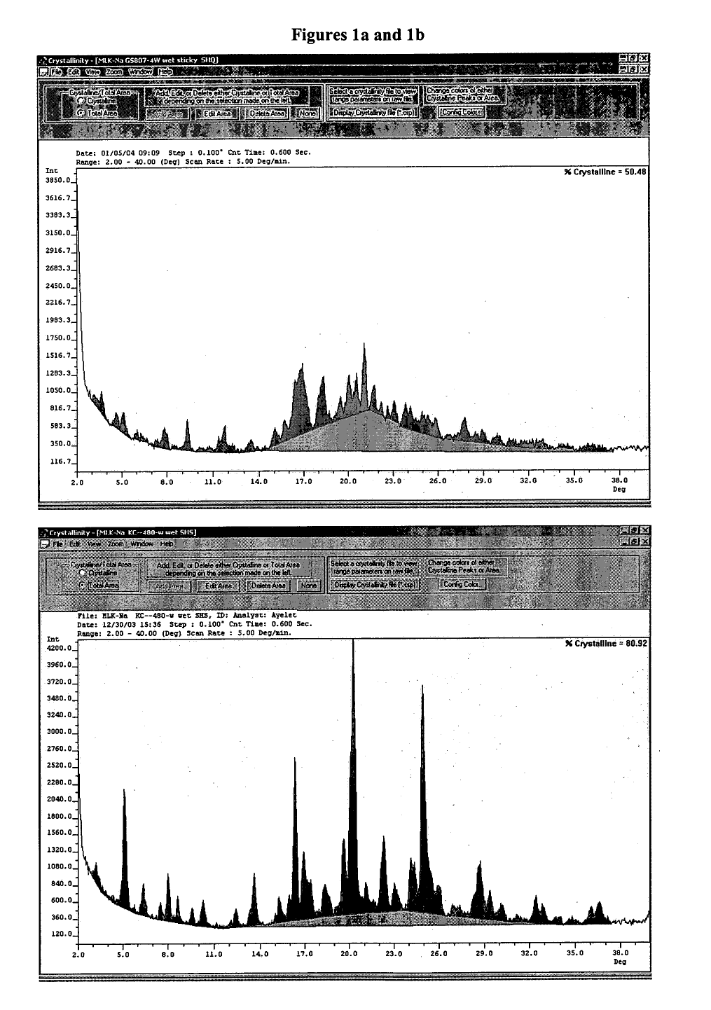

The present invention provides high crystallinity montelukast sodium. As used herein, the term “high crystallinity” means having a crystalline index of at least about 40%, more preferably at least about 60%, more preferably at least about 70%, and most preferably at least about 80%. The crystallinity index (CI) measures how much of a given sample includes the crystalline form as opposed to the amorphous form. The crystallinity index can be measured quantitatively from the X-ray powder diffractogram by comparing the area of the crystalline peaks (A_{C}) to the area under the halo-shaped amorphous peak (A_{A}). Thus, (A_{C}+A_{A}) equals the total scattered intensity. The crystallinity index is represented by the formula: CI=A_{C}* 100/(A_{C}+A_{A}). CI is estimated at ±5%, due to fluctuation in the baseline. FIG. 1 illustrates the areas A_{C} and A_{A} that are used for the crystallinity index calculation.

One embodiment of the invention provides a crystalline form of montelukast which has an X-ray powder diffraction pattern that is substantially free of peaks at 4.5 and 6.2±0.2 degrees two-theta. The term “free of peaks” as used herein means that the X-ray powder diffraction pattern is substantially flat in that no diffraction results in a peak characteristic of a crystalline structure.

Yet another embodiment of the invention provides a process for preparing high crystallinity montelukast sodium such as montelukast sodium having a crystalline index of at least about 40%, more preferably at least about 60%, more preferably at least about 70%, and most preferably at least about 80%. The high crystallinity montelukast sodium may have powder XRD pattern substantially free of peaks at 4.5 and 6.2±0.2 degrees two-theta. The process of preparing high crystallinity montelukast sodium comprises crystallizing the crystalline form from a solution of montelukast in a polar solvent and recovering the crystalline form. Preferably, the polar solvent is aprotic. More preferably, the polar solvent includes at least one of dimethyl carbonate, methyl isobutyl ketone, dichloromethane, dichloroethane, ethyl acetate, butyl acetate, isobutyl acetate, or water. The process may further include combining the solution with an anti-solvent, preferably a C₅ to C₁₂ hydrocarbon such as heptane or hexane. The process may further include maintaining the solution at room temperature until a precipitate forms. The process may further include cooling the solution.

Another embodiment of the invention provides crystalline montelukast sodium which is hydrate. Another embodiment of the invention provides crystalline montelukast sodium which is solvate. Crystalline montelukast has chemical, physical, and thermodynamic stability and is non-hygroscopic, as opposed to the amorphous form.

The present invention also provides crystalline forms of montelukast sodium named Forms A1, B2, B1, C, D, and E. The present invention also provides processes for preparing these crystalline forms. In another embodiment, the present invention provides pharmaceutical compositions containing these crystalline forms and also methods of treating respiratory diseases using the same.

One of skill in the art can identify a particular crystalline form by its X-ray powder diffraction pattern, taking into account the normal amount of experimental variation to be expected in the diffraction pattern.

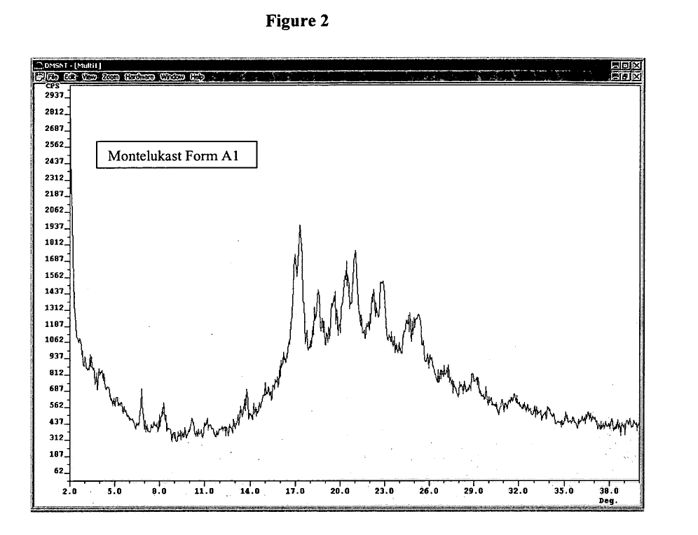

In one embodiment, the present invention provides crystalline montelukast sodium Form A1, herein defined as Form A1. Form A1 is identified by an X-ray powder diffraction pattern with peaks at 16.9, 17.2, 22.2, 22.7, and 25.2±0.2 degrees two-theta. Form A1 may be identified further by X-ray powder diffraction peaks at 18.5, 19.6, 20.4, and 21.0±0.2 degrees two-theta, as illustrated by FIG. 2.

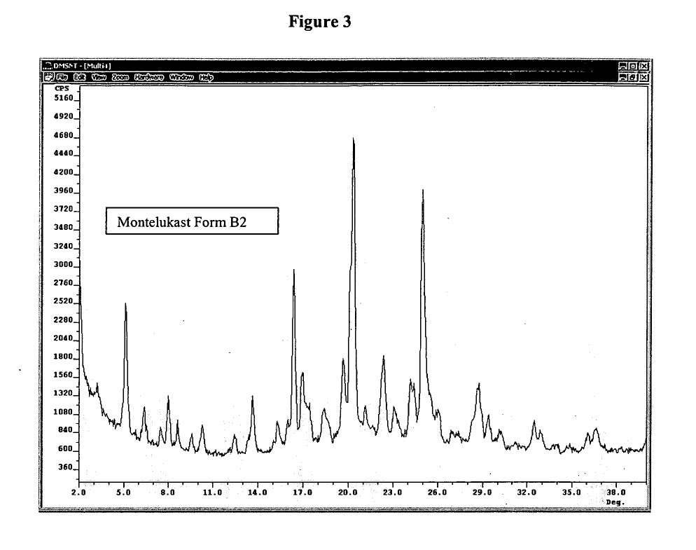

Yet another embodiment of the invention provides crystalline montelukast sodium Form B2, herein defined as Form B2. Form B2 is identified by an X-ray powder diffraction pattern with peaks at 5.1, 16.3, 17.0, 20.3, and 25.0±0.2 degrees two-theta. Form B2 may be identified further by X-ray powder diffraction peaks at 8.0, 13.6, 18.4, 19.7, and 22.3±0.2 degrees two-theta, as illustrated by FIG. 3.

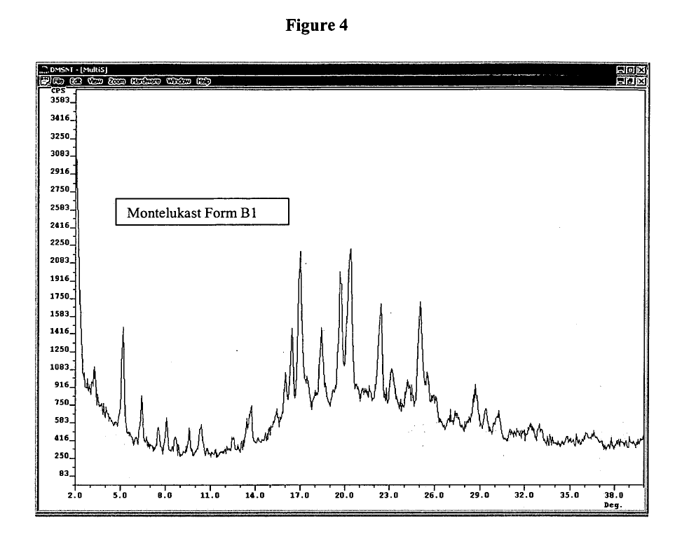

Another embodiment of the invention provides crystalline montelukast sodium Form B1, herein defined as Form B1. Form B1 is identified by an X-ray powder diffraction pattern with peaks at 5.3, 16.9, 19.6, 20.3, and 25.0±0.2 degrees two-theta. Form B1 may be identified further by X-ray powder diffraction peaks at 3.3, 18.3, and 22.3±0.2 degrees two-theta, as illustrated by FIG. 4.

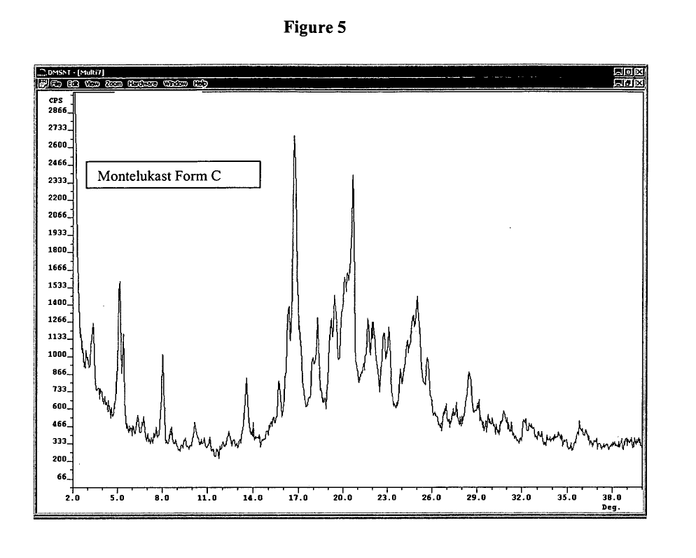

Yet another embodiment of the invention provides crystalline montelukast sodium Form C, herein defined as Form C. Form C is identified by an X-ray powder diffraction pattern with peaks at 5.2, 5.5, 16.7, 18.2, and 20.6±0.2 degrees two-theta. Form C may be identified further by X-ray powder diffraction peaks at 8.0, 13.5, 16.3, 19.4, and 23.1±0.2 degrees two-theta, as illustrated by FIG. 5.

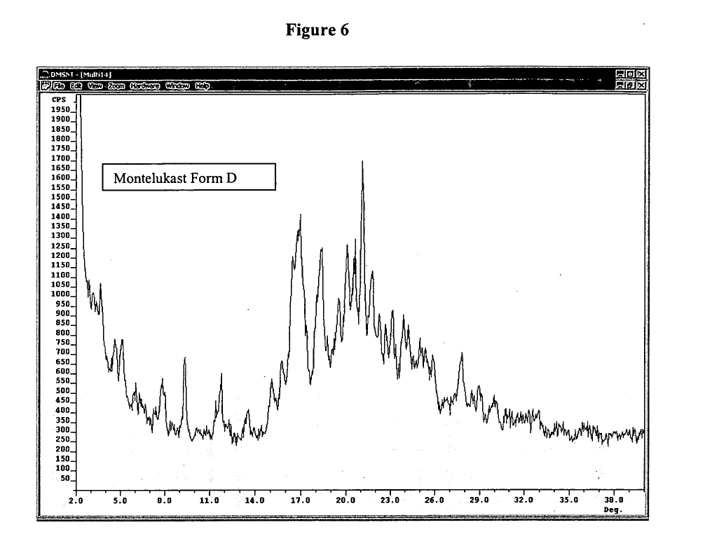

Another embodiment of the invention provides crystalline montelukast sodium Form D, herein defined as Form D. Form D is identified by an X-ray powder diffraction pattern with peaks at 11.8, 20.1, 20.6, 21.1, 21.8±0.2 degrees two-theta. Form D may be identified further by X-ray powder diffraction peaks at 9.3, 16.9, 18.3, 22.7, 23.1±0.2 degrees two-theta, as illustrated by FIG. 6.

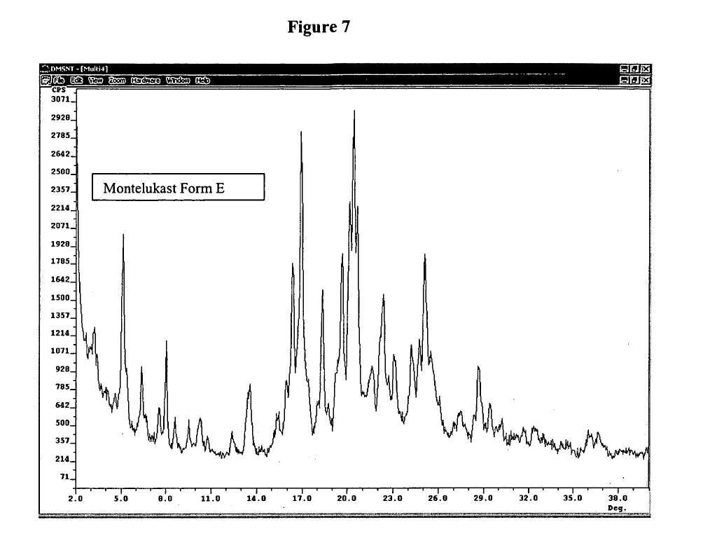

Yet another embodiment of the invention provides crystalline montelukast sodium Form E, herein defined as Form E. Form E is identified by an X-ray powder diffraction pattern with peaks at 16.9, 20.1, 20.5, 20.7, and 25.0±0.2 degrees two-theta. Form E may be identified further by X-ray powder diffraction peaks at 5.1, 6.4, 8.0, 16.5, and 18.4±0.2 degrees two-theta, as illustrated by FIG. 7.

The present invention also provides processes for preparing crystalline montelukast sodium. In one embodiment, the process for preparing crystalline forms of montelukast sodium includes the steps of crystallizing the crystalline form from a solution of montelukast in a polar solvent and recovering the crystalline form.

The solution is prepared by dissolving montelukast in a polar solvent. The starting material for the dissolving step may be any crystalline or amorphous form of montelukast sodium, including any solvates and hydrates. For processes in which montelukast sodium is dissolved into a solution, the form of the starting material is of minimal relevance since any solid state structure is lost in solution. For suspension and drying processes, the starting material may sometimes make a difference, as one of skill in the art would appreciate. Polar solvents for dissolution include aprotic solvents and include, but are not limited to, at least one of dimethyl carbonate (DMC), methyl isobutyl ketone (MIBK), dichloromethane (CH₂Cl₂), heptane, dichloroethane, ethyl acetate (EtOAc), butyl acetate (BuOAc), isobutyl acetate (iBuOAc), and water. Some embodiments utilize a polar solvent, which generally has a polarity greater than that of n-butanol. The amount of the solvent should be sufficient to dissolve the montelukast. One of ordinary skill in the art can easily determine the sufficient amount of the solvent. The process may further include combining an anti-solvent with the solution. Examples of anti-solvents include C₅ to C₁₂ hydrocarbons such as heptane and hexane. When the solvent is used with an anti-solvent, the combination is described as a ratio volume/volume.

Preferably, the crystallization step is performed with stirring. Stirring can be achieved by any means including, but not limited to, mechanical and magnetic means. The crystallization step can be performed at about 20° C. to about 25° C. (“room temperature” or “RT”) or at an elevated temperature of at least about 40° C., preferably about 55° C. to about 60° C. The crystallization step can be performed preferably for about 1 hour to about 72 hours. The crystallization step may further include facilitative measures known to one skilled in the art. For example, the crystallization step may further include cooling the solution, heating the solution, or adding an agent to induce precipitation.

Recovering the crystalline form, can be performed by any means known in the art including, but not limited to, filtration, centrifugation, and decanting. Preferably, the crystalline form is recovered by filtration. The crystalline form may be recovered from any composition containing the crystalline form and the solvent including, but not limited to, a suspension, solution, slurry, or emulsion.

The process may further include washing the crystalline form.

The process may further include drying the crystalline form. Drying can be performed under ambient or reduced pressure, though with some forms, a transformation to another form may occur.

In one embodiment, the invention provides processes for preparing crystalline montelukast sodium Form A1 including the steps of crystallizing the crystalline form from a solution of montelukast in dimethyl carbonate, and recovering the crystalline form. Preferably, the crystallization step further includes stirring the solution. The crystallization step can be performed preferably from about 1 to about 24 hours. Preferably, the crystalline form is recovered by filtration. The process may further include washing the crystalline form. The process may further include drying the crystalline form.

In one embodiment, the invention provides processes for preparing crystalline montelukast sodium Form B2 including the steps of crystallizing the crystalline form from a solution of montelukast in a C₃ to C₇ ketone or ester, and recovering the crystalline form. When an ester is used, to reduce solubility of the solution, an anti-solvent, preferably a C₅ to C₁₂ hydrocarbon such as heptane is added to the solution to precipitate the crystalline form. Preferred solvents include MIBK and MIBK with about 1% water by volume. Preferred solvents also include ethyl acetate, butyl acetate, or i-butyl acetate with heptane as an anti-solvent. Preferably, the crystallization step further includes stirring the solution. Preferably, the crystalline form is recovered by filtration. The process may further include washing the crystalline form. The process may further include drying the crystalline form.

In one embodiment, the invention provides processes for preparing crystalline montelukast sodium Form B1 including the steps of crystallizing the crystalline form from a solution of montelukast in dichloromethane and recovering the crystalline form. The process may further include combining the solution with an anti-solvent, preferably a C₅ to C₁₂ hydrocarbon such as heptane which induces crystallization. Preferably, the crystallization step further includes stirring the solution. Preferably, the crystalline form is recovered by filtration. The process may further include washing the crystalline form. The process may further include drying the crystalline form

In one embodiment, the invention provides process for crystallizing montelukast sodium Form C including the steps of crystallizing the crystalline form from a solution of montelukast in chlorinated hydrocarbon such as dichloroethane or dichloromethane, and recovering the crystalline form. The process may further include combining the solution with an anti-solvent, preferably a C₅ to C₁₂ hydrocarbon such as heptane which induces crystallization. Preferably, the crystalline form is recovered by filtration. The process may further include washing the crystalline form. The process may further include drying the crystalline form.

In one embodiment, the invention provides processes for crystallizing montelukast sodium Form D including the steps of dissolving montelukast in dichloromethane to form a solution, heating the solution, combining the solution with an anti-solvent, and recovering the crystalline form. The solution is heated to a temperature of at least about 40° C., more preferably at least about 50° C. The anti-solvent is preferably a saturated C₅ to C₁₂ hydrocarbon, more preferably heptane or hexane. Preferably, the crystallization step further includes stirring the solution. The crystallization step can be performed for about 24 to about 72 hours. Preferably, the crystalline form is recovered by filtration. The process may further include washing the crystalline form. The process may further include drying the crystalline form.

In one embodiment, the invention provides processes for crystallizing montelukast sodium Form E including the steps of crystallizing the crystalline form from a solution of montelukast in butyl acetate, and recovering the crystalline form. Preferably, the crystallization step further includes stirring the solution. Preferably, the crystalline form is recovered by filtration. The process may further include washing the crystalline form. The process may preferably further include drying the crystalline form.

The various forms are related in that drying of one form may lead to another form. Drying may be carried out under ambient or reduced pressure under heating. The temperature during heating is preferably about 40° C. to about 60° C., more preferably about 40° C. to about 50° C. The drying for a few days, such as about two days may be sufficient for transformation. In a preferred embodiment, drying is performed at 50C. overnight under vacuum, about 10-100 mm Hg. Drying of Form B2 results in Form B1 or form C, and drying of Form B1 results in Form C.

The various forms are related in that one form may transform to another while in an organic solvent, such as a slurry resulting from crystallization. Form B1, for example, transforms to Form C if stirred in an organic solvent for more than about 2 days, more preferably more than about 3 days. Such transformation occurs for example in a reaction mixture containing dichloromethane and optionally a C₅ to C₁₂ hydrocarbon such as heptane.

Many processes of the present invention involve crystallization out of a particular solvent. The term “crystallization” as used herein includes the dissolution of the starting compound to obtain a clear solution, and maintaining the solution for a period of time, with or without cooling or other inducement. The dissolution can take place at ambient temperature. One skilled in the art would appreciate that the conditions concerning crystallization can be modified without affecting the form of the polymorph obtained. For example, when mixing montelukast sodium in a solvent to form a solution, warming of the mixture may be necessary to completely dissolve the starting material. If warming does not clarify the mixture, the mixture may be diluted or filtered. To filter, the hot mixture may be passed through paper, glass fiber or other membrane material, or a clarifying agent such as celite. Depending upon the equipment used and the concentration and temperature of the solution, the filtration apparatus may need to be preheated to avoid premature crystallization.

The conditions may also be changed to induce precipitation. A preferred way of inducing precipitation is to reduce the solubility of the solvent. The solubility of the solvent may be reduced, for example, by cooling the solvent.

In one embodiment, an anti-solvent is added to a solution to decrease its solubility for a particular compound, thus resulting in precipitation. Another way of accelerating crystallization is by scratching the inner surface of the crystallization vessel with a glass rod. Other times, crystallization may occur spontaneously without any inducement. The present invention encompasses both embodiments where crystallization of a particular form of montelukast sodium occurs spontaneously or is induced/accelerated, unless if such inducement is critical.

As used herein, an anti-solvent is a liquid that when added to a solution of X in the solvent, induces precipitation of X. Precipitation of X is induced by the anti-solvent when addition of the anti-solvent causes X to precipitate from the solution more rapidly or to a greater extent than X precipitates from a solution containing an equal concentration of X in the same solvent when the solution is maintained under the same conditions for the same period of time but without adding the anti-solvent. Precipitation can be perceived visually as a clouding of the solution or formation of distinct particles of X suspended in the solution or collected at the bottom the vessel containing the solution.

One skilled in the art may also appreciate that the scope of the disclosure is not limited by the order of the additions in adding an antisolvent. For example, a solution may be added to an antisolvent or vice versa, though an embodiment may prefer one over the other. Usually crystallization is better when a solution is added to the antisolvent, but operationally it is often more convenient to add the antisolvent to the solution.

One embodiment of the invention provides pharmaceutical compositions containing the crystalline forms of montelukast sodium of the invention and methods of treating respiratory diseases using the same.

Pharmaceutical compositions of the present invention contain crystalline montelukast sodium such as one of those disclosed herein, or montelukast sodium purely amorphous, optionally in mixture with other form(s) of montelukast. Montelukast that is crystallized by the processes of the present invention is ideal for pharmaceutical formulation. In addition to the active ingredient(s), the pharmaceutical compositions of the present invention may contain one or more excipients. Excipients are added to the composition for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure-from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, montelukast and any other solid excipients are dissolved or suspended in a liquid carrier such as water, vegetable oil, alcohol, polyethylene glycol, propylene glycol, or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules.

Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention may include any of the aforementioned blends and granulates that were described with reference to tableting, however, they are not subjected to a final tableting step.

Methods of administration of a pharmaceutical composition for treating respiratory diseases, especially asthma, encompassed by the invention are not specifically restricted, and can be administered in various preparations depending on the age, sex, and symptoms of the patient. For example, tablets, pills, solutions, suspensions, emulsions, granules, and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously, or intraperitoneally. Suppositories may be administered into the rectum.

The amount of montelukast sodium contained in a pharmaceutical composition for treating respiratory diseases, especially asthma, according to the present invention is not specifically restricted, however, the dose should be sufficient to treat, ameliorate, or reduce the symptoms associated with the respiratory disease. The dosage of a pharmaceutical composition for treating respiratory diseases according to the present invention will depend on the method of use, the-age, sex, and condition of the patient. Typically, a dose is from about 2 mg to about 20 mg, with about 4 mg, 5 mg, or 10 mg of montelukast sodium being preferred. Preferred dosage forms include tablets, both chewable and non-chewable, and a granule.

Having described the invention, the invention is further illustrated by the following non-limiting examples.

### EXAMPLES

### Example 1

### Crystallizing Montelukast Sodium

Amorphous montelukast sodium salt (2 g) was dissolved in a solvent and stirred until a precipitate formed. Some solutions were stirred at room temperature; others were heated to the indicated temperature. The precipitate was recovered by filtration and washed with the solvent (5 mL) to obtain a wet sample. A portion of the wet sample was dried overnight in a vacuum at 50° C. at 10-50 mm Hg to obtain a dry sample. The wet and dry samples were analyzed by X-ray diffraction. The results are summarized on Table 1.

TABLE 1
Crystallizing montelukast sodium
Volume of solvent, including anti-solvent, is in mL per gram of
montelukast.

| | Vol. | | | XRD | | |
|---|---|---|---|---|---|---|
| First | (1 g/ | Temp. | Time | Sample | | |
| Solvent | mL) | (° C.) | (hrs) | wet/dry | Form | CI |
| DMC | 2.7 | 55 | 1 | w | A1 | 46% |
| DMC | 2.6 | 60 | 24 | d | A1 | 49% |
| MIBK | 10 | RT | 72 | w | B2 | 80% |
| | | | | d | B2 + | 66% |
| | | | | | B1 | |
| MIBK + | 7.5 | RT | 24 | w | B2 | 57% |
| 1% water | | | | d | B1 | 70% |
| by volume | | | | | | |
| MIBK | 2.6 | 60 | 24 | w | B2 | 78%-80% |
| MIBK | 2.6 | 60 | 72 | w | B2 | 80% |
| | | | | d | B2 + | 70% |
| | | | | | C | |
| CH₂Cl₂:Heptane | 7 | RT | 24 | w | B1 | 64% |
| (2:5) (vol/vol) | | | | d | C | 65% |
| CH₂Cl₂:Heptane | 14 | RT | 72* | W | C | 54% |
| (4:10) (vol/vol) | | | | | | |
| CH₂Cl₂:Heptane | 2.6 | 60 | 24 | w | D | 64% |
| (2:5) (vol/vol) | | | | | | |
| CH₂Cl₂:Heptane | 7 | 60 | 72 | w | D | 50% |
| (2:5) (vol/vol) | | | | d | D | 61% |
| CH₂Cl₂ 2 + 1 | 7 | RT | 24 | w | B1 | 64% |
| Hep. 5 + 1 | | | | d | C | 62% |
| (added in two | | | | | | |
| portions) | | | | | | |
| Dichloroethane | 5.3 | RT | 24 | d | C | 47% |
| EtOAc:Heptane | 5 | RT | 24 | w | B2 | 45% |
| (2:3) (vol/vol) | | | | d | B1 | 41% |
| BuOAc | 2.6 | 60 | 72 | d | E | 73% |

*The presence of these forms after long crystallization times points to a transformation of one form to another.

### Example 2

### X-ray Diffraction Analysis

The crystal forms were identified using an ARL Applied Research Laboratory (SCINTAG) powder X-ray diffractometer model X′TRA equipped with a solid state detector. The crystal samples were analyzed using a round aluminum sample holder with zero background and copper radiation of 1.5418 Å.

The crystalline index was calculated using the SCINTAG built-in software for crystallinity calculation.

TABLE 2
X-ray diffraction peaks for crystalline forms of montelukast sodium
Peaks are measured in degrees two-theta ± 0.2 degrees two-theta.
Peaks in bold are the most characteristic peaks.

| Form A1 | Form B2 | Form B1 | Form C | Form D | Form E |
|---|---|---|---|---|---|
| 16.9 | 5.1 | 3.3 | 5.2 | 9.3 | 5.1 |
| 17.2 | 8.0 | 5.3 | 5.5 | 11.8 | 6.4 |
| 18.5 | 13.6 | 16.9 | 8.0 | 16.9 | 8.0 |
| 19.6 | 16.3 | 18.3 | 13.5 | 18.3 | 16.5 |
| 20.4 | 17.0 | 19.6 | 16.3 | 20.1 | 16.9 |
| 21.0 | 18.4 | 20.3 | 16.7 | 20.6 | 18.4 |
| 22.2 | 19.7 | 22.3 | 18.2 | 21.1 | 20.1 |
| 22.7 | 20.3 | 25.0 | 19.4 | 21.8 | 20.5 |
| 25.2 | 22.3 | | 20.6 | 22.7 | 20.7 |
| | 25.0 | | 23.1 | 23.1 | 25.0 |

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used for guidance. All references mentioned herein are incorporated in their entirety.

1. A crystalline form of montelukast having a crystalline content of at least about 40% as area percentage XRD.

2. The crystalline form of claim 1, wherein the crystalline content is at least about 60%.

3. The crystalline form of claim 2, wherein the crystalline content is at least about 70%.

4. The crystalline form of claim 3, wherein the crystalline content is at least about 80%.

5. The crystalline form of any preceding claim, which has a powder XRD pattern substantially free of peaks at 4.5 and 6.2±0.2 degrees two-theta.

6. The crystalline form of any preceding claim, wherein the crystalline form is a hydrate or a solvate.

7. A process for preparing the crystalline form of any of claims 1-4, comprising:
a) crystallizing the crystalline form from a solution of montelukast in a polar solvent; and
b) recovering the crystalline form.

8. The process of claim 7, wherein the polar solvent is aprotic.

9. The process of claim 7 or 8, further comprising combining an anti-solvent with the solution.

10. The process of claim 9, wherein the anti-solvent is a C₅ to C₁₂ hydrocarbon.

11. The process of claim 10, wherein the anti-solvent is heptane or hexane.

12. The process of any of claims 7-11, wherein the polar solvent includes at least one of dimethyl carbonate, methyl isobutyl ketone, dichloromethane, dichloroethane, ethyl acetate, butyl acetate, isobutyl acetate, or water.

13. A crystalline form of montelukast sodium (Form A1) characterized by a powder XRD pattern with peaks at 16.9, 17.2, 22.2, 22.7, and 25.2 degrees two-theta ±0.2 degrees two-theta.

14. The crystalline form of claim 13, further characterized by peaks at 18.5, 19.6, 20.4, and 21.0±0.2 degrees two-theta.

15. A crystalline form of montelukast sodium (Form B2) characterized by a powder XRD pattern with peaks at 5.1, 16.3, 17.0, 20.3, and 25.0±0.2 degrees two-theta.

16. The crystalline form of claim 15, further characterized by peaks at 8.0, 13.6, 18.4, 19.7, and 22.3±0.2 degrees two-theta.

17. A crystalline form of montelukast sodium (Form B1) characterized by a powder XRD pattern with peaks at 5.3, 16.9, 19.6, 20.3, and 25.0±0.2 degrees two-theta.

18. The crystalline form of claim 17, further characterized by peaks at 3.3, 18.3, and 22.3±0.2 degrees two-theta.

19. A crystalline form of montelukast sodium (Form C) characterized by a powder XRD pattern with peaks at 5.2, 5.5, 16.7, 18.2, and 20.6±0.2 degrees two-theta.

20. The crystalline form of claim 19, further characterized by peaks at 8.0, 13.5, 16.3, 19.4, and 23.1±0.2 degrees two-theta.

21. A crystalline form of montelukast sodium (Form D) characterized by a powder XRD pattern with peaks at 11.8, 20.1, 20.6, 21.1, 21.8±0.2 degrees two-theta.

22. The crystalline form of claim 21, further characterized by peaks at 9.3, 16.9, 18.3, 22.7, 23.1±0.2 degrees two-theta.

23. A crystalline form of montelukast sodium (Form E) characterized by a powder XRD pattern with peaks at 16.9, 20.1, 20.5, 20.7, and 25.0±0.2 degrees two-theta.

24. The crystalline form of claim 23, wherein the crystalline form is further characterized by peaks at 5.1, 6.4, 8.0, 16.5, and 18.4±0.2 degrees two-theta.

25. A pharmaceutical composition comprising crystalline montelukast sodium according to any of claims 1-4, 13, 15, 17, 19, 21, or 23.
